(19) United States
(12) Patent Application Publication
(10) Pub. No.: US 2017/0231300 A1
(43) Pub. Date: Aug. 17, 2017

(54) Scarf Providing Arm Support

(71) Applicant: Ann Leslie, St. Louis, MO (US)

(72) Inventor: Ann Leslie, St. Louis, MO (US)

(21) Appl. No.: 15/530,518

(22) Filed: Jan. 24, 2017

Related U.S. Application Data
(60) Provisional application No. 62/388,944, filed on Feb. 11, 2016.

(51) Int. Cl. A41D 15/04; A41D 31/00; A61F 5/40; A41D 23/00

(52) U.S. Cl. CPC A41D 15/04; A41D 23/00; A41D 31/00; A61F 5/40

(57) ABSTRACT

An infinity scarf that can be worn about the neck of the wearer, and can be removed for application to the arms of the wearer, embracing the outer surfaces of parts of the upper arms of the wearer, and draping underneath of the lower arms, and the hands, held in position by the hands of the wearer, to function as a fully located arm support for the wearer during usage.

### CROSS REFERENCE TO RELATED APPLICATION

This regular letters patent application claims priority to the provisional application having Ser. No. 62/388,944, filed on Feb. 11, 2016.

### FIELD OF THE INVENTION

This invention relates generally to scarf, and more specifically an infinity scarf, of the type that is generally continuous in its circular configuration, and which incorporates some degree of elasticity, so that when the scarf is removed, and located about the upper and lower arms of the wearer, can function to provide arm support, in a rest position in front of the wearer, without the need for any other structural support for the arms and hands of the wearer.

### BACKGROUND OF THE INVENTION

There are numerous clothing items available in the prior art, in the category of scarves, or support type clothing for use by those with some impairment, or injury, as well known. For example, U.S. Pat. No. 6,435,185, to Schimpl, shows a travelers' comfort-wearable arm support device. This device surrounds the shoulders, the back of the wearer, fastens at the nape of the neck, and then loops around forwardly to provide support for the arms, as can be noted. This device is almost like a straight jacket, in its structure, and application.

Other prior art devices show various types of therapeutic muffs, such as the patent to Barry, U.S. Pat. No. 5,383,921.

A double arm sling, for use for rehabilitation purposes by the patient, is shown in U.S. Pat. No. 8,197,429, in the patent to Neseem.

Various types of items of clothing, such as scarves, and more particularly for what is identified in the clothing industry as an infinity scarf, can be seen in the patent to Mardirosian, U.S. Pat. No. 9,138,021.

A sling with pockets, in the patent to Heller, is shown in U.S. Pat. No. 7,841,997.

A shoulder brace, once again, for rehabilitation purposes, is disclosed in the patent to Charles, U.S. Pat. No. 5,018,513.

An upper extremity stabilizer, for use for restraining and immobilizing of the limbs or joints of a patent, can be seen in the patent to Mable, U.S. Pat. No. 5,095,894.

A fashion scarf that incorporates a hidden nursing cover can be seen in the patent to Leach, U.S. Pat. No. 9,003,565.

These are examples of prior art devices having some relationship to the concept of the current invention.

### SUMMARY OF THE INVENTION

This invention contemplates the formation, and transformation, of a scarf, and more particularly a scarf which is identified as an infinity or harmony scarf, that can be immediately transformed into an arm and hand support, particularly at those locations where jockeying for position for armrest support, such as on an airplane, in the theater, and at other locations, particularly where arm support must be shared with the adjacent occupants. More particularly, the concept of this invention is to provide and infinity scarf, that can normally be worn by the women, and even a man, for fashion purposes, for warmth, in its initial usage. Then, the scarf can be removed, and located about the upper arms, with each arm being located within the scarf, with the bottom segment of the scarf extending downwardly, at which location its lower edges can be grasped by the hands of the wearer, and therein provide full support for the arms, the forearms, the wrists, and the hands, without the need of any other supporting structure, of the type as previously identified. In this manner, there is no longer any jockeying for position particularly where the user may be located in adjacent seating, such as, for example, on the airline, where the space of the seating may be tight, having small width seats, and with the usage of this current invention, full arm support can be provided, without the need of any adjacent supporting structure. Hence, usage of this invention totally eliminates the need for the shifting of elbows, by adjacent passengers, trying to find some support upon the shared armrest, while traveling for an extended period of time.

The infinity scarf of this invention, preferable, has some elasticity, so that when it is initially introduced upwardly on the arms, or the upper arms are inserted within the scarf, the lower edges of the scarf while draping downwardly, will also by elastic contraction extend somewhat inwardly, so that those lower edges can be grasped by the hand of the opposite arm, pulled together, to provide full support under the forearms, and the hands of the user, during application.

This type of scarf can be of a preferred size that will allow for its application for the purposes of this invention, and the approximate size of the scarf may be about 18 inches in diameter, and approximately 12 inches in height, more or less, and which may be of various types of cloths, or knitted or crocheted cloth, in order to provide some stretch, for the purposes as previously described.

Thus, the concept of this invention is to provide a continuous scarf, that forms a circular configuration, that can be worn about the neck, and then transferred to the arms, for the support purposes as described herein. The scarf may be continuous, or it may be a length of material having ends, and the ends can be secured together by clasp, zipper, or other means of connection, including Velcro, when it is assembled for usage for supporting of the arms in position, as previously described.

It is, therefore, the principal object of this invention to provide an infinity scarf that can be worn for warmth and decorative purposes about the neck of the wearer.

Another object of this invention is to provide an infinity or continuous scarf that can be transferred for locating of the upper arms therein, in addition to the forearms, and then grasped at its lower edges by the hands, to provide a fully supporting structure for the arms during usage.

Another object of this invention is to provide an arm support that may be used during travel, while relaxing at the theater, being entertained at a movie, and allows the user to be fully rested, even while sleeping, such as when located on a airplane during travel.

Still another object of this invention is to provide an arm support that is very secure in holding the arms, forearms, and hands into a supportive position, without need for any other supporting structure.

Another object of this invention is to provide a scarf, formed as a tube, that can also be used for supporting the arms of the wearer.

Other objects and purposes of this invention may occur to those skilled in the art upon review of the summary of the invention as provided herein. Such variations, if within the spirit of this invention, are intended to be encompassed within the scope of any claims to patent protection issuing hereon.

### BRIEF DESCRIPTION OF THE DRAWINGS

In referring to the drawings:

FIG. 1 shows the infinity scarf of this invention being worn about the neck of the wearer;

FIG. 2 shows the infinity scarf of this invention as removed, and disclosing its annular configuration with some length;

FIG. 3 shows the scarf as applied to the upper arms of the wearer, in preparation for its securement of the forearms and hands therein;

FIG. 4 provides a sectional view, with the front portion of the scarf removed, to show how the scarf drapes downwardly from the upper arms, before the hands grasp its lower edges into a confining position;

FIG. 5 provides a side view of the embodiment as shown in FIG. 3;

FIG. 6 provides a front view of the scarf applied to the wearer with the hands grasping the lower edges of said scarf into a full arm supporting position; and

FIG. 7 shows a sectional view, with the front of the scarf being removed, to disclose how the hands grasp the lower edges of the scarf, to drape them under the forearms, into a full arm supporting position during usage.

### DESCRIPTION OF THE PREFERRED EMBODIMENT

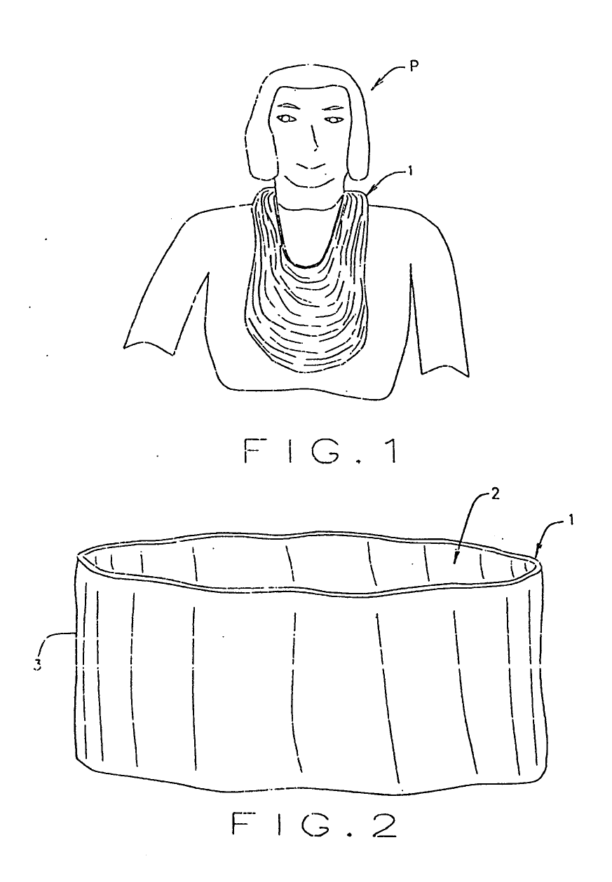

In referring to the drawings, and in particular FIG. 1, the concept of this invention comprising a scarf, otherwise known as an infinity or harmony scarf 1 is shown applied to the neck of the wearer, the person P as noted, and in this particular configuration, the scarf functions for both fashion purposes and to add comfort to the wearer, particularly during cooler temperatures. The scarf 1 as noted in FIG. 2, is generally of a continuous design, that has sufficient openness at its center 2 to provide adequate clearance for application of the scarf to the neck of the wearer, as previously noted. In addition, the scarf has sufficient height, as at 3, so that when it is used and applied for its alternative purpose, as an arm support, it will have adequate material to drape underneath the arms of the wearer, to add support thereat.

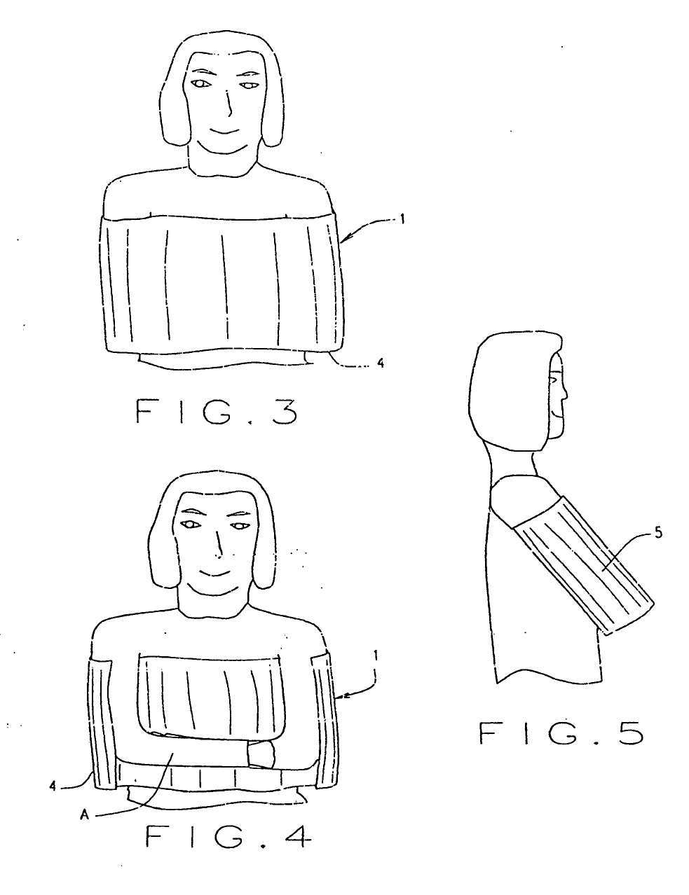

As noted in FIG. 3, once the scarf 1 is applied to the upper arms of the wearer, it drapes downwardly, for some length, as noted at 4, below the folded arms of the wearer, in preparation for usage as an arm support. As can be seen in FIG. 4, which is a sectional view, with the frontal portion of the scarf being removed, as noted, the folded arms A of the user, are noted. It can be seen that the scarf at its lower end 4, does extend downwardly, for some distance, below those folded arms.

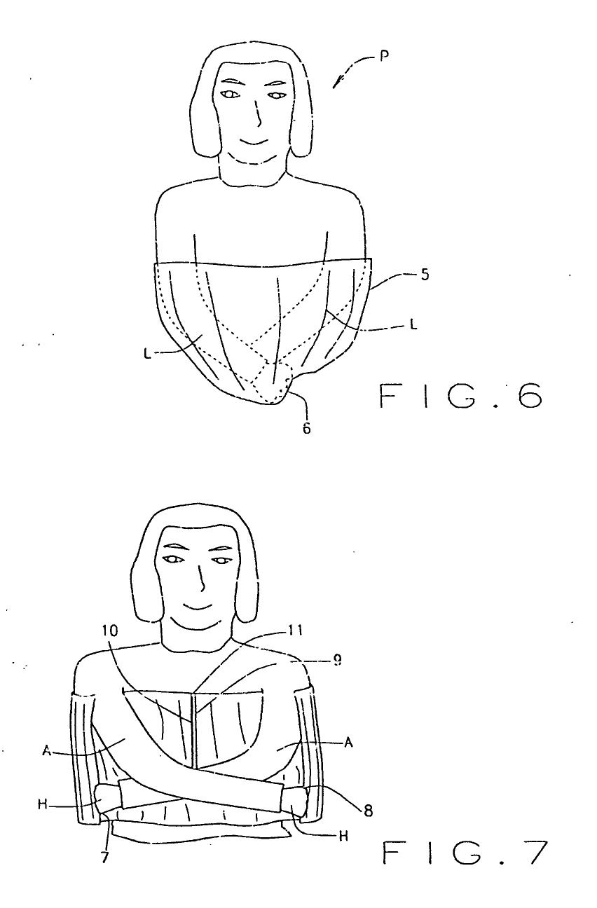

As noted in FIG. 5, the scar when draped around the arms of the user, fold about the outer surfaces of the arms, as can be seen at 5, in preparation for its application for supporting purposes. As can be seen in FIG. 6, which is a front view, the wearer grabs the lower edges, as at 6, of the scarf, at either side, and pulls those edges underneath of the lower arms L, and below the hands, so that the entire scarf now embraces the outer sides of the arms, as shown at 5, and likewise drapes under the grasping hands of the user, and in this position, functions to hold the arms inwardly against the body, and provides and under support for the lower arms and hands, to fully function as a supporting apparatus, as previously reviewed. Thus, when in this position, the user will have full arm support, does not need an adjacent arm rest for retention of the arms, holds the arms in a comfort position, having applicability when used for the various types of activities, as previously described, even for dosing, such as during travel, while being entertained, and for other occasions.

FIG. 7 provides a front view with the front section of the scarf 1 being opened, to show how each hand H will grasp the opposite lower edge of the scarf, as at 7 and 8, to hold the scarf under the lower arms, as at A, as previously described. In this manner, as can be readily understood, the scarf functions to provide full arm support, for the wearer, for a sustained period of time.

As previously alluded to, since the wearer might also be a male, who does not want to utilize an infinity scarf about his neck, the scarf may be formed of a length of the flexible scarf material, with its ends 9 and 10 being secured together by a zipper, as at 11, or by Velcro, clasp, buttons, or any other form of fastening means, that can hold the end edges of the scarf together, to form the continuous scarf for use for the purposes of this invention, which is to allow it to be installed and applied as an arm support, for the comfort of the user.

Variations or modifications to the subject of this invention may occur to those skilled in the art upon review of the development as provided herein. Such variations, if within the spirit of this invention, are intended to be encompassed within the scope of any claims to patent protection issuing upon this invention. The description of the invention in the preferred embodiment, and as depicted in the drawings, are primarily set forth for illustrative purposes only.

I claim:

1. An infinity or harmony scarf for wear for fashion and warmth purposes about the neck of the wearer, said scarf capable of removal from the neck of the wearer, and applied to the arms of the wearer, embracing the outer edges of said arms within the continuous scarf, and being draped under the lower arms and hands of the wearer, held in position thereat by means of the hands of the wearer, to provide full self support for the wearer's arms and hands without the need for any other supporting structure.

2. The scarf of claim 1, wherein the scarf is of continuous design.

3. The scarf of claim 1, wherein the scarf is a length of scarf material, said scarf material having a pair of ends, and the ends of the scarf material being secured by a fastener into a circular scarf configuration.

4. The scarf of claim 3, wherein said fastening means being one of a zipper, buttons, clasp, and hook and pile fastening means, to secure the ends of the scarf together into a circular configuration.

5. The scarf of claim 1, wherein said material is a flexible material.

6. The scarf of claim 1, wherein said scarf material is a resilient material.

7. The scarf of claim 1, wherein said material is formed of a textile.

8. The scarf of claim 1, wherein said material polymer.

9. An arm support, comprising a circular and continuous length of material formed into an annular form, said arm support applied to the arms of the wearer, embracing the outer edges of said arms within the continuous support, and being draped under the lower arms and hands of the wearer, held in position thereat by means of the hands of the wearer, to provide full self support for the wearer's arms and hands without the need for any other supporting structure
